(19) United States
(12) Patent Application Publication
(10) Pub. No.: US 2009/0280089 A1
(43) Pub. Date: Nov. 12, 2009

(54) Inhibition of the liver tropism of adenoviral vectors

(75) Inventors: Karim Benihoud, Paris (FR); Frédéric Vigant, Paris (FR); Michel Perricaudet, Ecrosnes (FR)

Correspondence Address: MORGAN LEWIS & BOCKIUS LLP, 1111 PENNSYLVANIA AVENUE NW, WASHINGTON, DC, 20004, US

(73) Assignee: Institut Gustave Roussy, Villejuif Cedex (FR)

(21) Appl. No.: 12/305,331

(22) PCT Filed: Jun. 19, 2006
(86) PCT No.: PCT/IB06/02493

§ 371 Date: May 4, 2009

(51) Int. Cl. A61K 35/76; C12N 15/64; A61P 1/16

(52) U.S. Cl. 424/93.2; 435/320.1

(57) ABSTRACT

The invention relates to the inhibition of liver tropism of adenoviral vectors, by replacement of the endogeneous HVR5 of hexon protein of said adenoviral vector with an heterologous polypeptide.

Adenovirus (Ad)-derived vectors are commonly used, in particular as gene therapy vectors, for instance for cancer therapy. However, the full potential of Ad gene transfer has not been fully realized because of the non-specific tissue-distribution of Ad vectors in vivo. Adenovirus receptors are expressed at low levels in some target tissues rendering them difficult to infect. On the other hand, both systemic and local administrations of these vectors lead to a liver transduction with a high risk of toxicity. Several attempts to abrogate Ad liver entry have been undertaken. They included mutations of specific residues of the capsid fiber protein to impair interactions with Ad5 natural receptors, Cocksackie and Adenovirus Receptor (CAR), integrins and heparan sulfate glycosaminoglycans (HSG), but also shortening of Ad5 fiber shaft or pseudotyping with other serotype's fibers. Though these approaches were more and less able to reduce liver tropism, they raised several concerns. Indeed, mutations or modifications of capsid proteins render the production of Ad vectors tricky while the use of fiber from other Ad serotypes furnishes Ad with the new fiber's entry pathway. Moreover, Ad liver entry may not only rely on known receptors-Ad interactions but also on Ad binding to blood factors (Shayakhmetov et al. J. Virol., 79, 7478-7491, 2005)

By studying the biodistribution in mice of Ad modified into hexon capsid protein, we unexpectedly observed that such a modification drastically reduced liver particle entry.

In the present study we stress a potential role of Ad hexon protein for liver entry in vivo. During bio-distribution studies involving a previously described lacZ recombinant Ad whose hypervariable region 5 (HVR5) of hexon protein was replaced by an αv-integrin binding RGD motif in place of the (AdHRGD) (Vigne et al. J. Virol., 73, 5156-5161, 1999) we observed surprisingly that AdHRGD was impaired for transgene expression in liver. To assess whether it was the RGD motif that redirected Ad to other organs or the HVR5 modification itself that led to diminution of transgene expression in liver, we constructed two lacZ-recombinant Ad whose hexon HVR5 was substituted with by a non-targeting peptide composed of a stretch of 8 or 24 Gly-Ala residues (AdH(GA)8 and AdH(GA)24), as shown in Table 1.

TABLE 1

| | | | | | | | Titer |
|---|---|---|---|---|---|---|---|
| | Upstream | | | | Length | Downstream | (10⁺¹² |
| Insert | sequence | Linker | Inserted Peptide | Linker | (AA) | sequence | pv.ml⁻¹) |
| wt | FFS₂₆₈ | — | TTEATAGNGDNLT | — | 13 | P₂₈₂KVVLYS | 10.0 ± 2.0 |
| RGD | FFS₂₆₈ | GS | DCRGDCF | GS | 11 | P₂₈₂KVVLYS | 4.4 ± 1.7 |
| (GA)₈ | FFS₂₆₈ | G | GGAGAGAG | LGG | 12 | P₂₈₂KVVLYS | 8.1 |
| (GA)₂₄ | FFS₂₆₈ | G | GGGAGAGGAGGAGGAGAGGAGAGA | LGG | 28 | P₂₈₂KVVLYS | 16.2 |

All these vectors are produced on conventional HEK-293 cells at levels comparable to that of a control Ad with unmodified capsid (AdHwt).

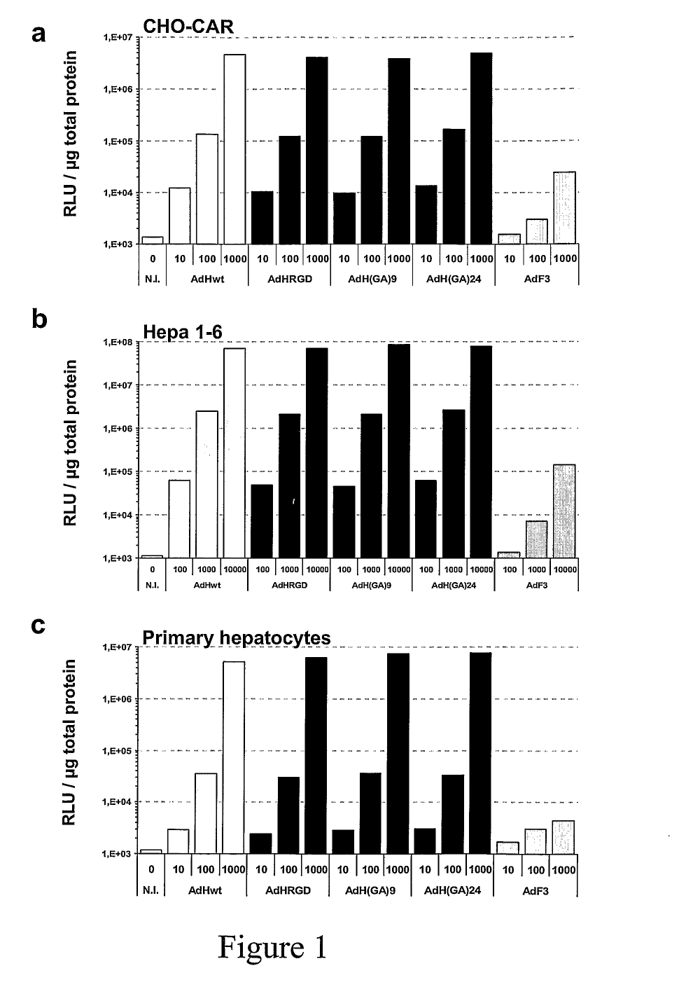

First, these Ad were analysed for their ability to transduce different cells lines. Plated cell monolayers of CAR-expressing cell line (CHO-CAR), of hepatocyte cell line (Hepa 1-6) or primary rat hepatocytes were infected with the different Ad at multiplicity of infection (MOI) of the different Ad. Twenty-four hours later, cells were lysed and β-Galactosidase (β-Gal) activity was measured using a chemiluminescent assay (Clontech, Palo Alto, Calif.) and expressed relative to protein content determined by the Bio-Rad Protein Assay. AdHwt, AdHRGD, AdH(GA)8, AdH(GA)24 transduced at the same level CHO-CAR, hepa 1-6 as well as primary hepatocytes whereas a previously described AdF3 (Vigne et al., Gene Therapy, 10, 153-162, 2003) pseudotype with an Ad3 fiber and that no longer binds to CAR receptor displayed a reduced transduction efficiency (see FIG. 1).

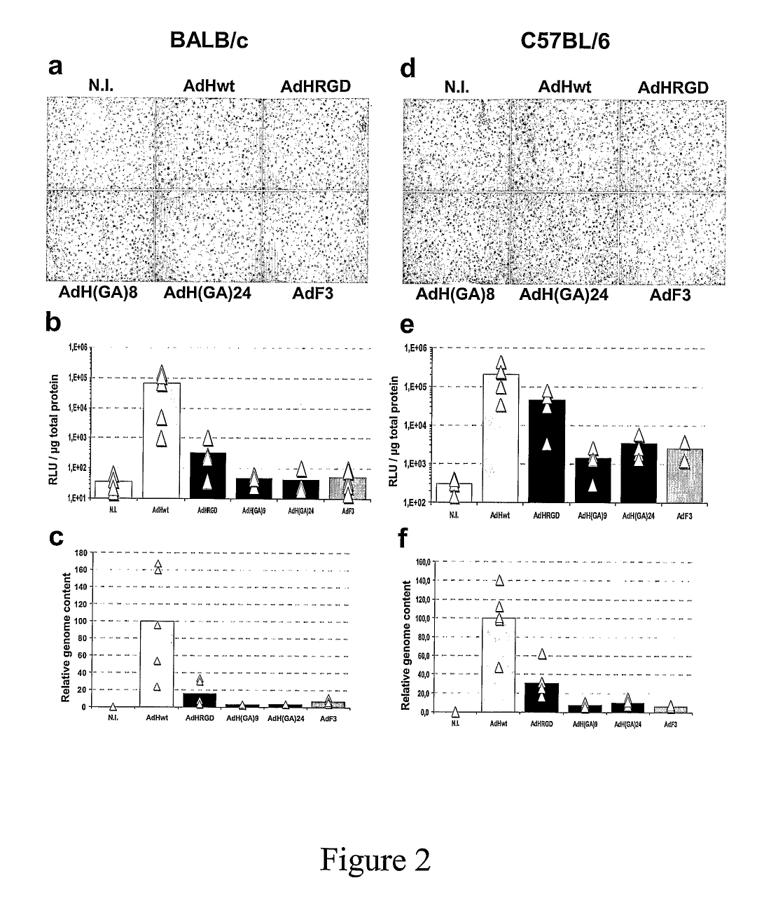

These results indicated that HVR5 modification per se does not modify Ad entry in vitro into hepatocytes and prompted us to assess liver gene transfer. BALB/c mice were intravenously (i.v.) injected with 10¹¹ viral particles (vp) of Adwt or capsid-modified Ad (AdHRGD, AdH(GA)8 and AdH(GA)24 and AdF3), sacrificed two days after and different pieces of liver were harvested for analysis of gene transfer by different techniques. While immunohistostaining of β-gal on liver sections indicated about 30% of hepatocyte transduction in AdHwt-injected mice, we observed a drastic reduction of hepatocyte labeling in all mice injected with Hexon-modifed Ad with only a few positive-hepatocytes (FIG. 2a), comparable to results obtained with AdF3 for which we reported in the past a strong impairment of liver transduction (Vigne et al. 2003, cited above).

Transduction efficiency was more accurately assessed by measurement of β-gal activity in liver lysates obtained from 50 mg of liver as described above. Thus, AdHRGD-, AdH(GA)8-and AdH(GA)24-injected mice exhibited a decrease of 99.5%, 99.9%, 99.9% in transgene expression as compared to AdHwt-injected mice (FIG. 2b).

To unravel whether this decrease was linked to a reduction in virus entry into liver, we extracted total DNA from 30 to 100 mg of liver using nucleospin Tissue Kit (MN) and we performed real-time quantitative PCR on 25 ng of total DNA to quantify viral DNA. Compared to Adwt, results displayed in FIG. 2c demonstrated a decrease of 84.5%, 97.3%, 96.9% and 93.0% in Ad DNA content in liver for AdHRGD, AdH(GA)8, AdH(GA)24 and AdF3, respectively.

To confirm our observation that modification of HVR5 region led to a profound reduction of Ad liver entry, we repeated the same experiment in a mice strain of other genetic background. Thus, in C57BL/6 mice, we observed a drastic reduction of β-gal expression as documented by immunohistochemistry (FIG. 2d) that was confirmed by a reduction in β-gal activity of 78% for AdHRGD and of 99.3% and 98.3% for both AdH(GA)8 and AdH(GA)24, respectively (FIG. 2e). This reduction in β-gal expression was linked to a 69.1%, 92.0% and 89.7% decrease of viral DNA content in AdHRGD-, AdH(GA)8- and AdH(GA)24-injected mice, respectively (FIG. 2f). These results clearly showed that HVR5 modification led to a reduction of virus entry compared to AdHwt. However, it should be noticed that the extent of this reduction varies depending of mice strain and the nature of the peptide inserted. Because HVR-modified Ad transduced efficiently primary hepatocytes, our results suggest that an unknown mechanism is occurring in vivo.

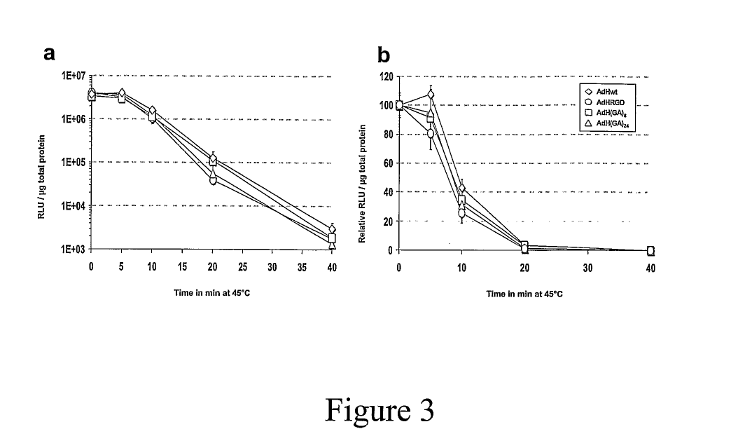

To rule out the possibility that HVR5 modifications affected the structural integrity of the virions, we compared thermostability of capsid-modified Ad to their wild-type counterpart. Viruses were incubated at 45° C. in serum free media for different time intervals before infecting CHO-CAR cells, β-gal expression was measured 24 h p.i. as reported before and expressed relative to protein content. We found that all HVR5-modified vectors showed similar stability to the unmodified virus (see FIG. 3). This suggested that incorporation of peptides of different length in HVR5 did not significantly affect the stability of Ad5, consistent with our results on virus production showing that modified viruses gave similar yields to unmodified Ad5 (see Table 1).

The invention thus provides a method for inhibiting the liver tropism of an adenoviral vector, wherein said method comprises replacing the endogenous HVR5 of hexon protein of said adenoviral vector with an heterologous polypeptide.

An “adenoviral vector” is an adenovirus which has been modified to carry a foreign gene into mammalian cells. Different types of adenoviral vectors are known in themselves, and can be modified according to the invention; the methods for modifying adenoviruses are also well-known in the art. For human therapy, the most commonly used adenoviral vectors are derived from type 2 or type 5 human adenoviruses (Ad 2 or Ad 5). It has however also been proposed to use adenoviral vectors derived from adenoviruses of animal origin, for instance canine (in particular CAV2), bovine, murine, ovine, porcine, avian, and simian origin (for recent review see for instance Volpers and Kochanek, J Gene Med., 2004 Feb.; 6 Suppl 1 :S164-71).

The “endogenous HVR5” herein refers to the naturally occurring hypervariable region 5 of the hexon protein, as found in a wild-type adenovirus. The position and length of said HVR5 may vary from one species of adenovirus to another. For instance, in wild-type Ad5 adenovirus, endogenous HVR5 corresponds to amino acids 269 to 281 of the hexon protein, and is flanked by a serine residue in position 268, and a proline in position 282; in wild-type Ad2 adenovirus, endogenous HVR5 corresponds to amino acids 280 to 293 of the hexon protein, and is also flanked by a serine residue in position 279, and a proline in position 294. HVR5 can be localised in other adenoviruses from the alignment of adenoviruses sequences, as disclosed for instance by Crawford-Miksza and Schnurr (J. Virol., 70, 1836-1844, 1996), or by Rux et al., (J. Virol., 77: 9553-9566, 2003)

The part of said endogenous HVR5 which is replaced by an heterologous polypeptide is preferably of at least 5 consecutive amino-acids, and up to the whole length of said HVR5.

An “heterologous polypeptide” herein refers to a polypeptide having a sequence other than the endogenous HVR5 sequence which is replaced. Preferably, said heterologous polypeptide has a sequence other than the HVR5 of a wild-type adenovirus. Preferably, said heterologous polypeptide is at least 5, and up to 35, more preferably up to 30, and advantageously up to 25 amino-acids long. Said heterologous polypeptide may be for instance a targeting peptide, such as those disclosed in PCT WO 00/12738, which allow to redirect the vector to a target tissue or organ other than the liver. Alternatively, it may also be a non-targeting peptide, i.e a peptide which is not expected to play a part in the targeting of the vector. Preferred non-targeting peptides are sequences consisting of amino-acids with short side chains such as Ser, and/or amino-acids with non-polar aliphatic side chains, such as Gly, Ala, Leu, Val, or Ile. Optionally, said heterologous polypeptide may also comprise at one or both ends, a spacer (or linker) comprising generally one to three amino acids. Preferred amino acids for the spacer include Gly, Ser, or Leu.

“Inhibiting the liver tropism of an adenoviral vector” refers to reducing the entry of said vector into liver cells in vivo of at least 70%, preferably at least 75%, and by order of increasing preference, at least 80%, 85%, 90%, or 95%, when compared with the corresponding adenoviral vector having an endogenous HVR5.

The invention also relate to the use of an adenoviral vector wherein at least a part of the endogenous HVR5 of the adenoviral hexon protein has been replaced by an heterologous polypeptide, for preparing a composition whose liver tropism is inhibited, for gene therapy in vivo.

Said composition can be a composition for systemic administration. It can also be used advantageously for local administration, for instance intratumoral administration: even if a part of the administered vector escapes from the tumor, it will not be captured by the liver.

The present invention also relates to adenoviral vectors wherein at least a part of the endogenous HVR5 of the adenoviral hexon protein has been replaced by an heterologous polypeptide, in particular a non-targeting peptide, as defined above.

Said adenoviral vectors may also comprise additional modifications, outside the HVR5, allowing to redirect the vector to a specific target tissue or organ.

The adenoviral vectors modified according to the invention can be used in any of the usual applications of adenoviral vectors, except those wherein it is intended to deliver a nucleic acid of interest to the liver.

FIG. 1: Hexon-modified Ad5 gene transfer in vitro.

ChO-CAR (A), Hepa 1.6 (B) or freshly isolated rat hepatocytes (C) were infected with increasing MOI of AdHwt, AdHRGD, AdH(GA)₈ or AdH(GA)₂₄ or PBS (N.I.) encoding β-Gal. Twenty four hours later cells were lysed and β-gal activity measured. Experiments were done twice in duplicate and representative results are shown here.

FIG. 2: Gene transfer in liver following systemic delivery of hexon-modified adenoviruses.

C57BL/6 (a, b, c) or BALB/c (d, e, f) mice aged of 8 to 16 weeks were i.v. injected with 10¹¹ VP of lacZ recombinant Ad (AdHwt, AdHRGD, AdH(GA)₈ or AdH(GA)₂₄) or PBS (N.I.). Forty-eight hours later, mice were sacrificed and livers harvested. βal expression was assessed either by immunohistochemistry performed on paraffin section (a, d, original magnification×100) or by a chemiluminescence-based enzymatic assay (b, e). Total DNA was extracted from liver fragments and viral DNA content was measured by Real-Time PCR was performed (c, f, One of two experiment is shown, n=4-5/group; means± S.D. shown, * P<0.05 and ** P<0.01).

FIG. 3: Thermostabilities of Hexon-modified Ad5 vectors.

Aliquots of 10³ vp per cell of AdHwt (?), AdHRGD (O), AdH(GA)₈ ( ) or AdH(GA)₂₄ (?) were incubated at 45° C. for different time intervals and then used to infect CHO-CAR cells. Results are presented as the percentages of βgal activity detected, 24 h after infection, in cells infected with heat-treated viral sample with respect to βgal activity determined in the cells infected with unheated virus (100%). Each symbol represents the cumulative mean +/− SD of duplicate determinations. Some error bars depicting SDs are smaller than the symbols.

1. A method for inhibiting the liver tropism of an adenoviral vector, wherein said method comprises replacing the endogenous HVR5 of hexon protein of said adenoviral vector with an heterologous polypeptide.

2. (canceled)

3. A method for preparing an adenoviral vector for inhibiting liver tropism comprising replacing at least a part of endogenous HVR5 of an adenoviral hexon protein with an heterologous polypeptide therefore obtaining an adenoviral vector.

4. A method of inhibiting liver tropism comprising administering an adenoviral vector to an animal comprising at least part of endogenous HVR5 of an adenoviral hexon protein replaced with an heterologous polypeptide.
